(19) United States
(12) Patent Application Publication
(10) Pub. No.: US 2015/0173398 A9
(43) Pub. Date: Jun. 25, 2015

(54) METHOD FOR USING VINASSE

(71) Applicant: Weber Ultrasonics GmbH, Karisbad-Ittersbach (DE)

(72) Inventor: Siegmar Vogel, Berlin (DE)

(73) Assignee: Weber Ultrasonics GmbH, Karlsbad-Itterbach (DE)

(21) Appl. No.: 14/238,016

(22) PCT Filed: Jul. 23, 2012
(86) PCT No.: PCT/EP2012/064367

§ 371 Date: Feb. 10, 2014

(30) Foreign Application Priority Data
Aug. 9, 2011 (EP) PCT/EP2011/063706
Mar. 26, 2012 (DE) 10 2012 204 807.3
Apr. 24, 2012 (DE) 10 2012 206 700.0

(51) Int. Cl. A23K 1/06; C12N 1/12; A01K 61/00; C12N 1/10

(52) U.S. Cl. CPC A23K 1/06; C12N 1/10; C12N 1/12; A01K 61/005

(57) ABSTRACT

In order to thoroughly solve existing vinasse problems, in particular in production of bioethanol from plant raw materials, a method is proposed for producing feedstuffs from crustaceans of the genus Artemia (brine shrimp) or Copepoda (oar-footed crustaceans) or Daphnia (water fleas) or from microbes in the form of rotifers or protozoa, the method being characterized in that the crustacean/microbe cultures are fed at least partially with vinasse, preferably thin vinasse, in particular from bioethanol production, and with yeasts contained in the vinasse. At the same time, a method for processing vinasse is provided, the method accordingly being characterized in that the vinasse is used as feed for crustacean/microbe cultures and/or for algae cultures in order to at least partially decompose the yeasts contained in the vinasse. In connection with this, it is also proposed that process heat produced during vinasse production, in particular in the course of producing bioethanol, is used to heat water for the cultivation of crustaceans/microbes and/or algae in order to use the thermal energy content of the vinasse in a useful manner.

### BACKGROUND

The present invention relates to a method according to the preamble of patent claim 1 for producing feedstuff from crustacea of the genus Artemia or Copepoda (oar-footed crustacea) or Daphnia (water fleas), from rotifers (rotifera) or from eukaryotes (protozoa).

In addition, the invention relates to a method for processing vinasse, particularly in the course of bioethanol production.

Furthermore, the present invention relates to a method for producing feedstuff from crustacea of the genus Artemia or Copepoda (oar-footed crustacea) or Daphnia (water fleas), from rotifers (rotifera) or from eukaryotes (protozoa) and/or for producing raw materials from algae.

Vinasse is fermentation residues as a byproduct in bioethanol production which has developed in recent decades into a worldwide environmental problem. The regular untreated disposal of vinasse to rivers and in agriculture has, due to the chemical composition thereof, and the biological pollution, led to enormous environmental destruction. This includes, inter alia, local fish death and an over acidification or over fertilization of agricultural soils due to the uncontrolled dumping of vinasse. The problem is all the more serious since in ethanol production, depending on the starting material, between 4 and 151 of thin vinasse are generated for each liter of bioethanol produced.

The ethanol industry is searching intensively for approaches to solutions in order to deal with the vinasse problem. For instance, currently in many countries of the world, the vinasse produced is concentrated by energy-intensive evaporation of liquids, in order in this manner to decrease the costs of further transport. The end product can then be used as animal feed supplement or as fertilizer. The additional costs arising in this case are enormous. In addition, the high process heat content of vinasse, the temperature of which is about 60° C. on formation, remains unutilized.

### SUMMARY

The object of the invention is to specify alternative methods for utilizing vinasse in order to manage the environmental problems described above and to decrease the transport and energy costs associated to date with the disposal of vinasse. In addition, a sustainable contribution to the topic of nutrition is intended to be made, in particular also in emergent and developing countries.

The object is achieved by one or more of the methods according to the invention Advantageous developments of these methods are described below and in the claims, the wording of which is hereby explicitly incorporated by reference into the description, in order to avoid as far as possible unnecessary text repetition.

A method according to the invention for producing feedstuff from crustacea of the genus Artemia or the genus Copepoda (oar-footed crustacea) or the genus Daphnia (water fleas), from rotifers (rotifera) or from eukaryotes (protozoa) is characterized in that the Artemia/Copepoda/Daphnia/rotifers/protozoa cultures are fed at least in part with vinasse, preferably thin vinasse, in particular from bioethanol production, and with the yeast cells present therein. The cultured crustacea or rotifers/protozoa may then be used for feedstuff, in particular for fish culture, for example after appropriate processing (pelletizing).

Another method according to the invention for processing vinasse, particularly in the course of bioethanol production, is characterized in that the vinasse, preferably after appropriate treatment, is used as feed for Artemia/Copepoda/Daphnia/rotifers/protozoa cultures and/or for algal cultures, in order to remove at least in part the yeast cells present in the vinasse. Dumping of vinasse may be avoided in this manner.

A further method according to the invention for producing feedstuff from crustacea of the genus Artemia or the genus Copepoda or the genus Daphnia, from rotifers (rotifera) or from eukaryotes (protozoa) and/or for producing raw materials from algae is characterized in that, in the vinasse production, particularly in the course of bioethanol production, process heat generated is used to heat water or the like for the Artemia, Copepoda, Daphnia, rotifer, protozoa and/or algal cultures. Culturing is thereby made possible even where climatic conditions would otherwise be unsuitable.

In this manner, in the context of the present invention, for the first time an overall concept is proposed which avoids disposal of vinasse in the conventional sense and considers it in every respect a valuable raw material which may be used in many ways, but principally for the production of nutrients for or in aquacultures, whereby the environmental problems outlined above are very largely eliminated.

Firstly, in the context of the present invention, provision is made for using the vinasse virtually directly as feedstuff for Artemia/Copepoda/Daphnia/rotifer/protozoa cultures and/or for algal cultures. In order not to endanger these, provision can be made in this context for elevating the pH of the vinasse and preferably additionally to buffer it before feeding. This can be achieved, in particular, by adding carbocalk (CaCO3). Ca(OH)2 or other bases may also be used as alternatives to carbocalk.

A further aspect of the present invention accordingly relates to a method for processing vinasse, in which the vinasse is used as feed for Artemia/Copepoda/Daphnia/rotifer/protozoa cultures and/or for algal cultures, which is an extremely environmentally acceptable type of utilization/disposal of vinasse.

In order to be able to deal with the large amounts of vinasse produced in the course of bioethanol production of up to 50 m3 per hour in the case of a typical plant, a preferred development of the methods according to the invention provides that the Artemia/Copepoda/Daphnia/rotifers/protozoa are maintained in closed intensive cultures. Alternatively however, a culture in semi-open or open cultures is also possible.

Artemia, due to its nutritional characteristics, has proved in recent decades an outstanding feedstuff not only in freshwater but also in saltwater fish cultures. Due to the increasing demand for Artemia, the market prices have also stabilized at a high level. The growing market for aquacultures requires novel concepts and strategies in feeding of the stock, as proposed in the present case. In particular, the worldwide production of bioethanol or vinasse is sufficient to cover the worldwide protein demand for aquacultures, including all growth estimates in the aquaculture sector. An important step would thereby be taken against the overfishing of the world's seas, and the production of fishmeal could be at least severely restricted. In the past, in this connection, pellets from fishmeal factories were frequently used. The great ecological problems associated herewith such as, e.g., the overfishing of the seas and the processing of excessively small raw material from juvenile sea creatures are adequately known and demand a general rethinking among the operators of such aquacultures.

Artemia, due to its biological characteristics (high multiplication factor) and the possibility of maintenance in intensive culture, is one variant which, due to a lack of other technical approaches, to date is usually only widespread in open systems and only in climatologically warm areas. In addition, the feeding of intensive cultures of Artemia to date is a non-negligible cost factor.

This applies analogously to Copepoda (oar-footed crustacea), which show a good reproducibility on feeding with vinasse. This applies both to pelagic (open-water) and to benthic (on or in at the sea floor-dwelling) types of Copepoda. Copepoda form the most species-rich group of the crustacea and are in terms of mass the largest proportion of marine plankton (zooplankton) which renders them suitable for use as fish or shrimp feed. Moreover, comprehensive studies exist on the use of Copepoda as rearing feed in the fish industry, where the high nutritional value is particularly striking

Daphnia (water fleas) also, particularly Daphnia magna (large water fleas), rotifers and protozoa can be readily cultivated when feeding with vinasse according to investigations by the applicant.

Therefore, by the proposed use of vinasse as feedstuff for intensive Artemia, Daphnia and Copepoda cultures or for culturing rotifers/protozoa, in addition to the problems already mentioned many times in the utilization/disposal of vinasse, the abovementioned problems in connection with the operation of intensive Artemia/Daphnia/Copepoda/rotifer/protozoa cultures may also be bypassed or eliminated.

Due to the use proposed according to the invention of the process waste heat in the vinasse production, the intensive Artemia/Copepoda cultures described may also be achieved in those climate zones where Artemia, Daphnia, rotifer, protozoa or Copepoda culturing would otherwise not be lucrative, for example, due to water temperatures that are too low or an energy requirement that is too high.

Although it is to be expected that the selling prices specifically for Artemia, Daphnia and Copepoda, but also for rotifers or protozoa, in the course of the proposed intensive maintenance, will have a trend to develop in the downward direction, compared with the costs otherwise to be employed for vinasse disposal, a non-negligible financial advantage still results.

Within the scope of the proposed intensive maintenance of Artemia/Copepoda/Daphnia (crustacea) or rotifers/protozoa (microbes) and/or algae, high-performance filtration systems are needed in order to destroy potentially hazardous microbes in the process water and in addition prevent the occurrence of rotting processes which could otherwise endanger in particular the Artemia, Copepoda, Daphnia, rotifer, protozoa or algal cultures. In this connection, in the scope of an extremely preferred development of the methods according to the invention, it is proposed that the (process) water used in the crustacean cultures, microbial cultures and/or algal cultures is exposed to ultrasound for sterilization, preferably with ultrasound in the megasound range (f≧500 kHz). In this manner, microbiological impurities of the process water may also either be eliminated directly or disrupted in such a manner that they can readily be eliminated by further process water treatment steps optionally connected downstream.

Another development of the methods according to the invention, in this connection, provides that the water used in the crustacean/microbial cultures and/or the algal cultures is alternatively or additionally irradiated with short-wave light, preferably in the ultraviolet spectral range, for sterilization. Most preferably, for this purpose, light having a wavelength in the range from 1 to 380 nm, preferably about 250 nm, is used.

Continuous monitoring of the process water quality of the intensive crustacean/microbial/algal cultures, associated with a corresponding automation, makes it possible to process even very large volumetric flow rates, as occur in an industrial method for utilizing vinasse.

Vinasse, on account of its composition, may be filtered only with difficulty, or not at all, which is due, in particular, to the presence of large amounts of yeast cells in the size range from only 5 to 10 μm. The use of nanofiltration or reverse osmosis, due to said organic pollution with yeast cells, can be carried out only using additional techniques. In the past these could be implemented only by complex industrial processes. The present invention here can provide a remedy by supplying the vinasse waste product to the novel utilization described.

The application and exploitation of marine algal cultures and of the resultant algae is varied and as such is not subject matter of the present invention. The algae generated can, however, in turn be fed to crustacean/microbial cultures. In addition, methods have become known with which valuable raw materials or motor fuels, such as biodiesel, or even hydrogen, can be obtained from algae. All of these methods can be used in conjunction with the present invention.

Algal cultures, just like crustacean/microbial cultures, are a growing market. The exploitation of fertilizer salts from vinasse in production plants for maritime algae is therefore a considerable improvement in the production of marine algae and in addition contributes to improved utilization of the overall vinasse potential.

The process waters from said algal production can be filtered and separated by reverse osmosis. It has proved to be particularly advantageous that the salt lyes produced in this case can be fed back into the crustacean/microbial production, in order there to ensure optimum process parameters.

For purification, the process water from the crustacean/microbial production in the course of a particular development of the invention is purified by microfiltration through a membrane filter having a pore size preferably <1 μm. Generally, the use of pore sizes of 2 μm and smaller is possible. The membrane filter is here advantageously located in the culture container and is operated in outside-in mode. This filter may be comprised of bundles of membrane hollow fibers which are arranged within a stable retaining or frame structure. The membranes can thereby be removed as a whole from the container in order to be cleaned. The cleaning is preferably conducted by exposure to ultrasound.

Due to the pore size mentioned and the large filter surface, the differential pressure at the filter surface is low such that there is no risk of the smallest organisms (particularly Artemia, Daphnia or Copepoda, but also rotifers and protozoa) being sucked in and harmed.

In a biofilter (biologically active filter) advantageously connected downstream of the membrane filter, ammonium and other organic molecules in the permeate are decomposed by bacteria. Additionally or alternatively, the process water (permeate) may also be used as fertilizer for the algal production.

As stated above, the vinasse formed in ethanol production has a temperature of about 60° C. This heat potential remains unexploited to date, but in the scope of the present invention, it can be utilized for the crustacean/microbial production process and also for algal culture, which in each case require temperatures of about 26° C. Therefore, the total market is also opened up in temperate zones, and so the nutrient requirement of aquacultures can be covered directly, in particular by bioethanol factories, without relatively great expenditure on energy and costs for transport even in temperate zones.

### BRIEF DESCRIPTION OF THE DRAWINGS

Further properties and advantages of the present invention result from the following description of exemplary embodiments with reference to the drawing.

FIG. 1 shows a schematic drawing of the process flow relating to the overall process of the vinasse treatment proposed in the scope of the present invention;

FIG. 2 shows a simplified drawing of the overall process according to FIG. 1;

FIG. 3 shows a special modification of the method and the process sequence according to FIG. 2;

FIG. 4 shows schematically a crustacean production system, as can be used for carrying out the method according to the invention; and

FIG. 5 shows schematically another crustacean/microbial production system, as can be used for carrying out the method according to the invention.

### DETAILED DESCRIPTION OF THE PREFERRED EMBODIMENTS

The following description relates by way of example to the utilization/disposal of vinasse, as occurs as a byproduct in ethanol production from biological raw materials, for example sugar cane. This type of ethanol production is also termed, in the scope of the present description, “bioethanol production”. The invention, however, is not limited in principle to the use of vinasse generated in such a manner.

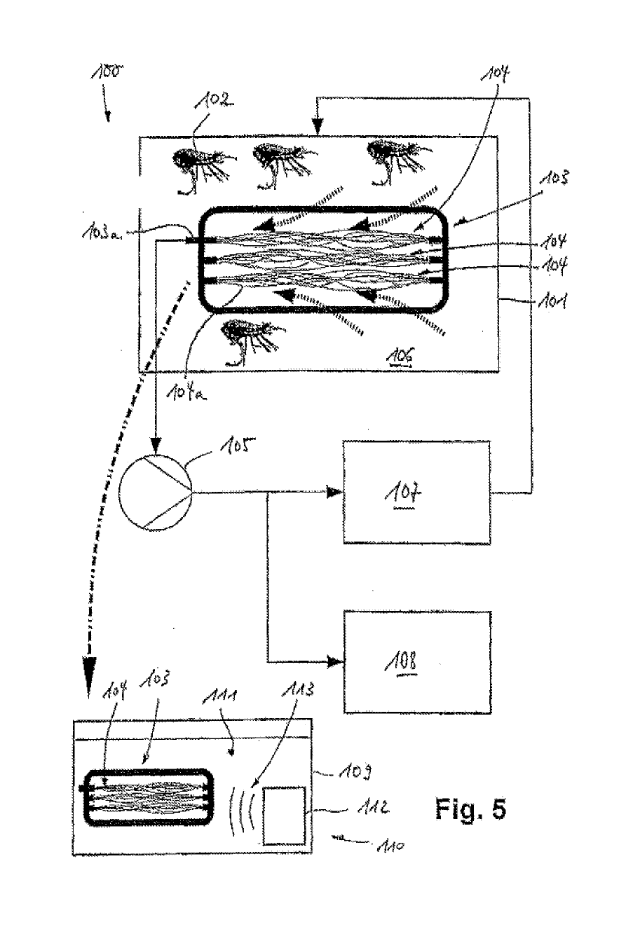

Furthermore, the following description is largely restricted—with the exception of FIG. 5—to the culturing of Artemia. The invention is generally also applicable, however, in the context of culturing crustacea of the genus Copepoda (oar-footed crustacea) or Daphnia (water fleas) and in the context of culturing rotifers (rotifera) or eukaryotes (protozoa) or common zooplankton.

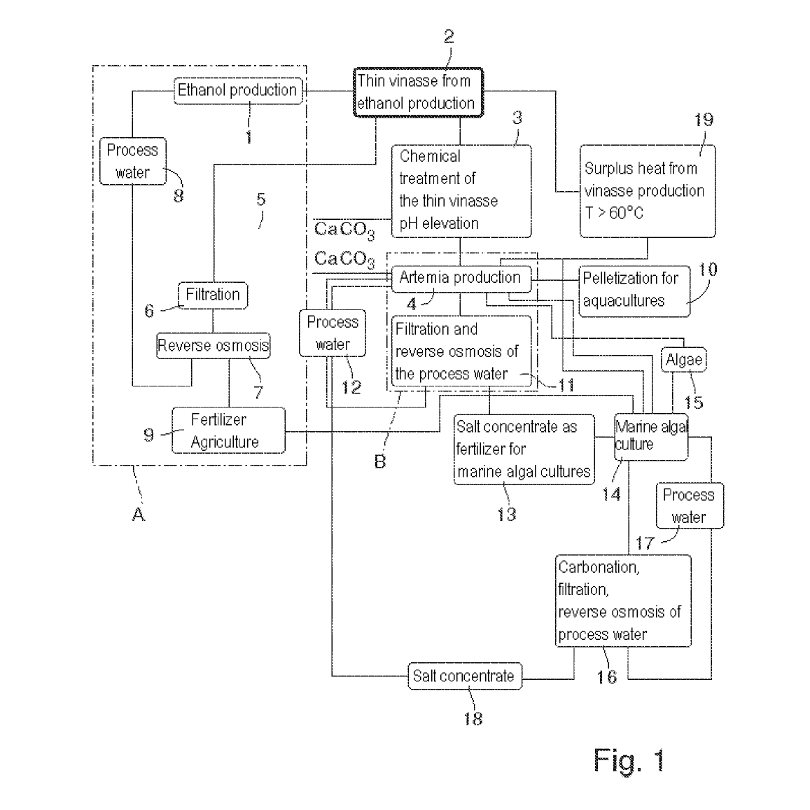

FIG. 1 shows schematically the process flow in an overall process for vinasse treatment (disposal and utilization), as can be achieved in the course of the present invention.

Reference sign 1 denotes the actual ethanol production, in which vinasse is produced in the form of what is termed thin vinasse, as byproduct or waste product, which is indicated at reference sign 2. The amount of vinasse produced is relatively high and, in a typical plant for production of bioethanol from sugar cane, can be up to 50 m3 per hour, which represents corresponding problems in disposal, but also opens up corresponding possibilities in utilization.

The present invention now proposes to use the vinasse as feedstuff for Artemia cultures, in order in this manner to produce feedstuff from the cultured Artemia crustacea. For this purpose, the vinasse is first chemically prepared at reference sign 3, wherein, in particular, the pH thereof is elevated from typically about 4.5 to 8, in order not to endanger the Artemia. After said preparation at reference sign 3, the vinasse is used at reference sign 4 for Artemia production, that is to say is added to corresponding Artemia cultures as feedstuff.

The additive added in method step 3 for elevating the pH of the thin vinasse can be, in particular, carbocalk (CaCO3). The CaCO3—as already explained—may be used either in step 3 for the chemical preparation of the thin vinasse or in step 4 directly in the Artemia production—in the latter case, in order there to effectively counteract overacidification of the culture environment. The vinasse, after a filtration 6 and optionally reverse osmosis 7, is separated into process water 8 on the one hand and vinasse salts 9 on the other, wherein said vinasse salts 9 can then be used in particular as fertilizer in agriculture. The process water 8 can be fed back again to the ethanol production 1, in such a manner that to this extent a closed circuit is produced. The resultant vinasse salts 9 may also alternatively be further utilized in the scope of the invention, which will be considered further in more detail hereinafter.

The actual yield of the Artemia production 4 in the form of harvested Artemia crustacea is provided at reference sign 10 for use in aquacultures, for example for fish culture, in the form of pellets, flakes or the like. As those skilled in the art acknowledge, however, the use of the yield of Artemia production 4 is in no way restricted to the uses described above by way of example on the basis of reference sign 10.

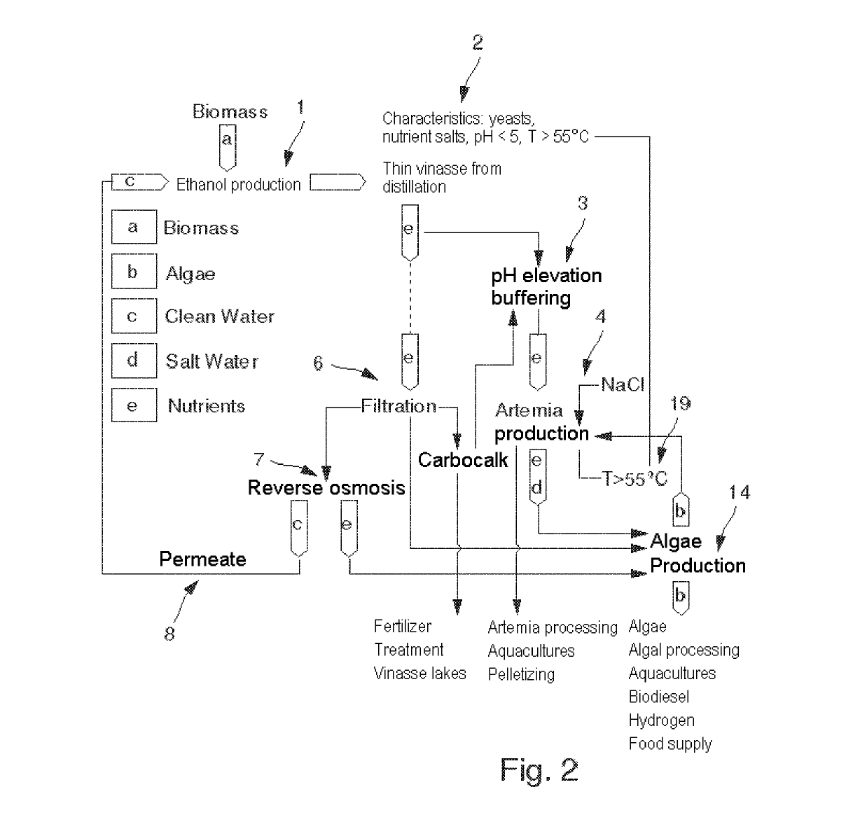

The overall system for Artemia production according to method step 4 including downstream filtration with reverse osmosis treatment of the process water 11 is characterized in FIG. 1 by the reference sign B (dot-dash line) and is described in still more detail below on the basis of FIG. 2.

The process water itself can, according to method step 12, be fed back to the Artemia production 4.

The salt concentrate 13 arising in the reverse osmosis of the process water 11 of the Artemia production 4 may be used—optionally after chemical treatment—as fertilizer for marine algal cultures 14 that deliver corresponding algae 15 as utilizable yield, which can either be utilized directly (e.g. for production of biodiesel) or, in the scope of the present invention, again can be fed as nutrient to the Artemia production 4. As has already been stated above, there is also the possibility of using the fertilizer salts 9 arising in the scope of method A for fertilizing the marine algal cultures 14.

In connection with the marine algal cultures 14, there is the possibility of carbonating the resultant liquid (waste) materials, filtering them and treating them by reverse osmosis (reference sign 16), wherein, in particular, the process water 17 can be fed back to the algal cultures 14.

The salt concentrate 18 arising in method step 16 in the reverse osmosis can be fed back to the Artemia production 4 together with the process water 16 described above, in order to guarantee the Artemia crustacea optimum growth conditions.

To round off the overall process described above according to FIG. 1, in the scope of the present invention, it can additionally be provided that the waste heat 19 produced in the generation of thin vinasse 2 in the scope of the ethanol production is made utilizable by suitable heat-exchange appliances in the scope of the Artemia production 4 and/or in the marine algal cultures 14. This can be achieved in that, for example, halls or buildings in which said cultures are colonized are heated in order to create an optimum growth environment. There is additionally, or alternatively, also the possibility of inexpensively warming the process water used 12, 17 to an optimum growth temperature by utilizing therefor the surplus heat 19 from the vinasse production.

Accumulating carbocalk may be utilized in order to eliminate past “environmental abuses”, such as for neutralizing so-called vinasse lakes, where the vinasse arising in the production of ethanol has been temporarily stored or disposed of, or for neutralizing overacidified soils.

A preferred implementation of the overall process of vinasse treatment described above provides incorporating, subsequently to method step 2, upstream of method steps 3 and 6, a rate controller or fractionation in order to subdivide the thin vinasse produced optionally respectively into a first amount of the filtration 6 and/or into a second amount of the chemical treatment in step 3 for subsequent use in the Artemia production 4—according to the instantaneous need.

FIG. 2 shows an alternative simplified presentation of the overall process from FIG. 1, wherein the same reference signs denote the same or equivalent process steps. The letters “a” to “e” denote material streams of biomass (a), algae (b), freshwater (clean water) (c), salt water (d) and nutrients (e).

According to FIG. 2, thin vinasse 2 is in turn formed from the biomass which is fed in the framework of the ethanol production 1, which thin vinasse is distinguished by the yeasts and nutrient salts present therein at a pH of less than 5 and a temperature of at least 55° C. or above. The chemical treatment already described with reference to FIG. 1 in step 3 (elevation of the pH and corresponding buffering) is followed by the feed of vinasse to the Artemia production 4. Alternatively, at reference sign 6, a filtration followed by a reverse osmosis treatment 7 can be carried out, whereupon the treated process water is fed as permeate 8 back to the ethanol production 1. The carbocalk optionally produced in the filtration 6 can either be reutilized at reference sign 3 or used as fertilizer or for treatment of vinasse lakes.

The Artemia production 4 delivers feedstuffs for aquacultures, for example for fish culture, as described above. This is preferably connected with pelletizing or other processing of the Artemia produced, as stated in FIG. 2.

The salt water and the remaining (vinasse) nutrients resulting from the Artemia production 4 are used at reference sign 14 for algal production. The algae produced can be used or further processed in many ways, for example for producing motor fuels or for producing foods. There are even algal cultures known which can be used for producing hydrogen, which correspondingly is likewise within the scope of the present invention. As can be further recognized, in particular, from FIG. 2, the algae produced can also in turn be fed to the Artemia production 4 as a nutrient.

The nutrients obtained in the reverse osmosis 7 may likewise be used for the algal production 14. The same applies to the residues of the filtration 6.

As likewise further illustrated in FIG. 2, the waste process heat from ethanol production 1 in the form of the heat contents of the vinasse 2 can be used at reference sign 19 for covering at least in part the heat requirement of the Artemia production 4 and/or of the algal production 14.

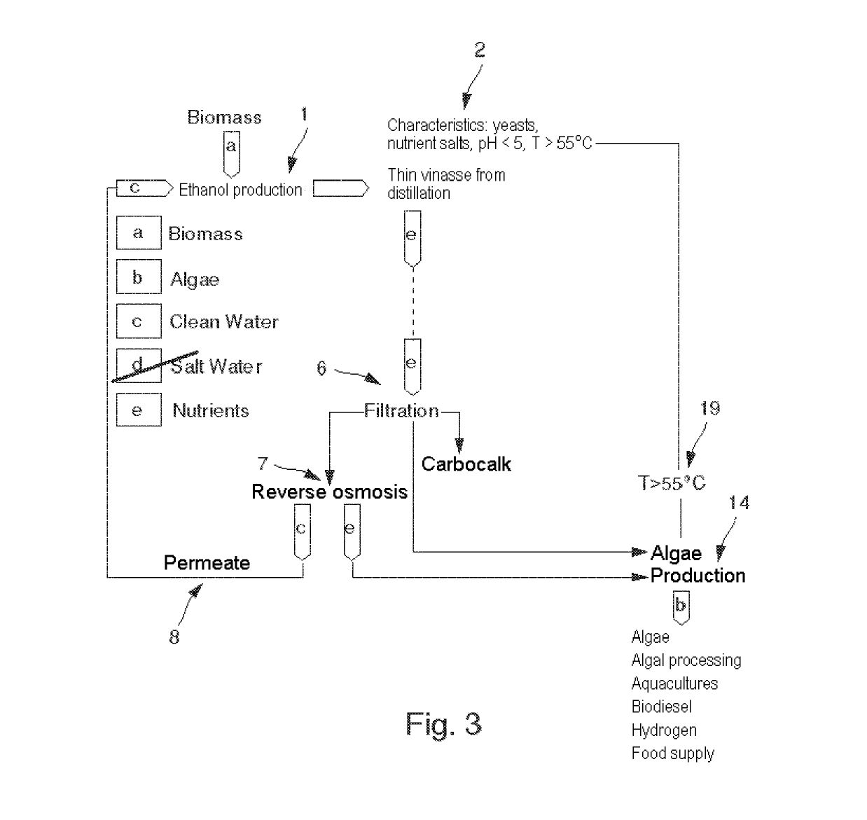

FIG. 3 shows an again simplified configuration of the process sequences according to FIG. 2. Here also, the same reference signs designate the same or equivalent process steps.

According to the presentation in FIG. 3, the Artemia production is dispensed with. After filtration 6 of the vinasse 2 has been completed, carbocalk is provided (cf. FIG. 2) and the supplying of the algal production 14 takes place.

A reverse osmosis 7 downstream of the filtration 6 in turn delivers treated process water 8 for the ethanol production 1 and nutrients for the algal production 14.

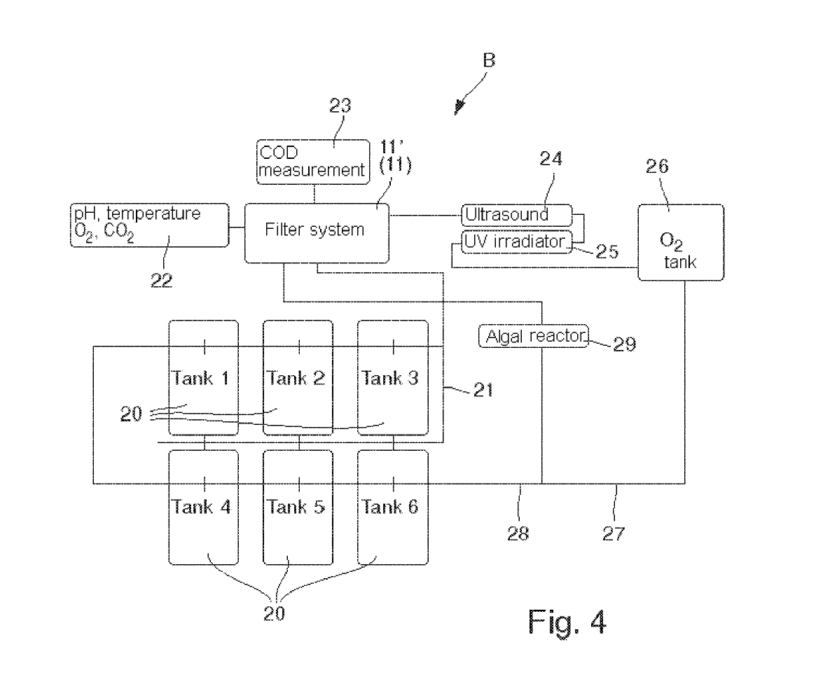

FIG. 4 shows schematically, and in detail, an Artemia production system, as may be used in the scope of the present invention and has been described above with reference to FIG. 1 at reference sign B.

The system shown in FIG. 4 for Artemia production according to reference sign 4 in FIG. 1 comprises a series of culture tanks or tanks 20, depending on size or dimensioning of the system. The tanks 20 are filled with salt water, in order to culture therein crustacea of the genus Artemia (also called in German “Salzkrebschen” or “Salzwasserkrebse” [brine shrimp]), which is known to those skilled in the art per se. The Artemia are fed at least in part with chemically treated thin vinasse as has already been described in detail above with reference to FIG. 1 (cf. there method steps 3 and 4).

Vinasse is protein- and nutrient-rich and is outstandingly suitable, after corresponding elevation and buffering of the pH, for feeding to Artemia. In particular, vinasse contains large amounts of yeast cells of a size of only about 5 to 10 μm, which cannot be removed by filtration, but can serve the Artemia as a nutrient and thus can be at least in part eliminated.

The wastewater or process water polluted with residues from Artemia production or remaining constituents of the vinasse passes via a line 21 via suitable conveying means (which are not shown) to a filter system 11′, which substantially corresponds to the reference sign 11 in FIG. 1. First measuring means 22 for determining pH, temperature, oxygen and CO2 content of the process water are in active connection with the filter system 11′. In addition, second measuring means 23 are connected to the filter system 11′, which measuring means 23 are designed for carrying out a COD measurement. The abbreviation COD is taken to mean chemical oxygen demand, which is taken to mean the amount (volume-related mass) of oxygen which is required for complete oxidation of the organic and inorganic materials in the wastewater. This is adequately known, for example, from the municipal and industrial wastewater treatment. Further water constituents which, in this connection, are advantageously measured—also in the framework of the present invention—are ammonium, total nitrogen, free and total chlorine, nitrate and phosphorus. Corresponding measuring instruments are adequately known to those skilled in the art.

Furthermore, the system has at reference sign 24 ultrasound means which are designed to disrupt and/or to eliminate certain pollutions of the process water from the Artemia production by ultrasound. The ultrasound means 24 comprise, in particular, suitable ultrasonic oscillators or transducers together with corresponding control/supply electronics, which are not shown explicitly, however, in FIG. 4 for reasons of clarity. In principle, all types of known ultrasonic oscillators can be used in this connection. A preferred frequency range in operation of the ultrasonic oscillators is in the megasound range (f≧500 kHz) in order to eliminate in this manner, in particular, bacteriological pollutions of the process water (killing by destruction of cell membranes or the like).

A UV irradiation unit 25 is assigned to the ultrasound means 24, which UV irradiation unit is likewise provided and designed to eliminate or kill biological pollutions of the process water by exposure to short-wave light radiation, preferably in the range of 1-380 nm, preferably about 250 nm.

Downstream of the UV irradiation means 25 is connected an oxygen tank 26 which has the function of replacing oxygen that is outgassed during the ultrasound treatment, in order to guarantee an optimal Artemia culture. The process water thus treated passes via lines 27, 28 back into the tanks 20.

As those skilled in the art acknowledge, UV treatment and ultrasound treatment need not proceed successively or in separate devices, but can also be performed substantially simultaneously or at the same site.

In addition, the system additionally comprises an algal reactor 29 which is connected between the filter system 11′ and the process water feed lines 27, 28 (cf. reference sign 30). The algal reactor 29 likewise serves for freeing the process water of the Artemia production from certain pollutants and waste materials, which is already sufficiently known from algal effluent treatment plants for wastewater purification. Such algal reactors are also known under the names algal photobioreactor for carbon dioxide fixation or for algal biomass production.

Alternatively, or in addition, algae can be specifically produced by the algal reactor 29. The algae thus obtained can be used either as food, for example again for the Artemia production (cf. reference signs 4 and 15 in FIG. 1) or for generating biodiesel, as already described.

The use of ozone (O3) for sterilizing the process water is likewise possible, if a harmful action on the Artemia culture overall can be excluded.

The present invention makes possible, in particular, the culture of Artemia and therefore the utilization of vinasse on an industrial scale of the type of intensive culture. In this connection, the treatment and sterilization of the process water produced in the Artemia culture is a particular challenge, wherein a key position is accorded to the ultrasound means 24 according to FIG. 4. Without the disruption achievable thereby of the in particular biological pollutions of the process water, the method would not be able to be put into practice or only with difficulty, or be restricted in throughput and yield only to small amounts, which would not bear any relation to the amounts of vinasse produced, in particular, in bioethanol production.

FIG. 5 shows schematically another crustacean production system 100, which may be used for carrying out the method according to the invention. In the actual culture container 101, Copepoda are cultured (not shown to scale at reference sign 102 by way of example) by using vinasse as described. Within container 101 is arranged a framework element 103 made from pipeline sections, which comprises a number of fiber bundles 104a, of which single fibers 104a are comriosed of hollow fibers and function together as membrane filters (pore size ≦2 μm, preferably ≦1 μm). At reference sign 103a, process water 106 is sucked out (which is symbolized by dashed arrows) of the container 101 by means of a conveying device (pump) 105 and is purified in the context of an outside-in filtration by the membrane filter hollow fibers 104a. The resulting permeate, which is contaminated with ammonium and other organic molecules, proceeds from the pump 105 either to a biofilter 107, where it is further treated by bacteria, or it is used at reference sign 108 as fertilizer for production of (marine) algae, as has already been indicated above. The permeate purified at 107 then arrives back at the container 101.

For cleaning the membrane filter hollow fibers 104a, the framework element 103 together with the fiber bundles 104 is removed from the container 101 (dashed-dotted arrow) and placed in the tank 109 of an ultrasonic cleaning device 110, which has been filled with a cleaning medium (fluid) 111. An ultrasonic transducer 112 of the ultrasonic cleaning device 110 subjects the framework element 103 to ultrasound 113 to free the membrane filter hollow fibers 104a of contamination. The framework element 103 can then be reused in the container 101.

In the scope of the present invention, an overall process is proposed for the first time which can comprehensively counteract the vinasse problems described.

1. A method for producing feedstuff from crustacea of the genus Artemia or Copepoda or Daphnia or from rotifers or protozoa microbes, comprising feeding crustacean/microbial cultures at least in part with vinasse from bioethanol production, and with yeast cells present therein.

2. A method for processing vinasse comprising using the vinasse as feed for crustacean cultures of at least one of the genus Artemia, Copepoda, or Daphnia, for microbial cultures for culturing rotifers or protozoa, as nutrient for algal culture in order to remove at least in part yeast cells present in the vinasse.

3. The method as claimed in claim 2, further comprising elevating a pH of the vinasse before the feeding of the at least one of Artemia, Copepoda, Daphnia, rotifers or protozoa.

4. The method as claimed in claim 2, further comprising the at least one of Artemia, the Copepoda, the Daphnia, the rotifers, the protozoa or the algae in closed intensive cultures.

5. The method as claimed in claim 4, further comprising exposing water used in the crustacean cultures, in the microbial cultures, or the algal cultures to ultrasound for sterilization.

6. The method as claimed in claim 5, further comprising exposing the water used in the crustacean cultures, in the microbial cultures or the algal cultures to short-wave light for sterilization.

7. The method as claimed in claim 1, further comprising carbonating process water from the crustacean/microbial cultures and separating the process water from foreign matter, by at least one of additional filtration or reverse osmosis.

8. The method as claimed in claim 7, wherein resultant salts or salt lyes are used as fertilizer for algal cultures.

9. The method as claimed in claim 8, wherein the cultured algae are used as feedstuff for the crustacean/microbial cultures or as raw material or for further processing.

10. The method as claimed in claim 8, process water from the algal cultures is carbonated and separated from foreign matter, by at least one of additional filtration or reverse osmosis.

11. The method as claimed in claim 10, wherein at least one of the resultant salts or salt lyes are fed back into the crustacean/microbial cultures.

12. The method as claimed in claim 1, wherein process water from the crustacean/microbial cultures is filtered through membrane filters which are arranged in a culture container of the crustacean/microbial cultures.

13. The method as claimed in at least claim 12, wherein membrane filter hollow fibers are used for the filtration, having a pore size <2 μm.

14. The method as claimed in claim 13, wherein the filtration is carried out in outside-in mode, that permeate is led away outwards from the culture container, and the permeate is subsequently purified, or the permeate is used as fertilizer for algal cultures.

15. The method as claimed in claim 14, wherein the membrane filters used for the filtration are removed from the culture container as part of a retaining structure which is removable as a whole from the culture container, and that the membrane filters removed are then cleaned in an ultrasonic cleaning device by exposure to ultrasound.

16. A method for producing feedstuff from crustacea of the genus Artemia or Copepoda or Daphnia or from microbes in the form of rotifers or protozoa or for producing raw materials from algae, in the vinasse production in the course of bioethanol production, comprising using process heat generated to heat water for the crustacean, microbial or algal culture.

17. (canceled)
